(19) United States
(12) Patent Application Publication
(10) Pub. No.: US 2019/0267114 A1
(43) Pub. Date: Aug. 29, 2019

(54) DEVICE FOR PRESENTING SEQUENCING DATA

(71) Applicant: GARVAN INSTITUTE OF MEDICAL RESEARCH, NEW SOUTH WALES (AU)

(72) Inventors: Mark COWLEY, New South Wales (AU); Velimir GAYEVSKIY, New South Wales (AU)

(73) Assignee: GARVAN INSTITUTE OF MEDICAL RESEARCH, NEW SOUTH WALES (AU)

(21) Appl. No.: 16/335,992

(22) PCT Filed: Jan. 25, 2017
(86) PCT No.: PCT/AU2017/050055

§ 371 Date: Mar. 22, 2019

(30) Foreign Application Priority Data
Sep. 22, 2016 (AU) 2016903841

(51) Int. Cl. G16B 45/00; G16B 30/20; G16B 50/10; G06F 16/28; G06F 16/22

(52) U.S. Cl. CPC G16B 45/00; G16B 30/20; G06F 16/2282; G06F 16/284; G16B 50/10

(57) ABSTRACT

This disclosure relates to a device for presenting whole genome sequence data. A file system stores a first file comprising short variant data and a second file comprising long variant data. A database stores variant data as data records. A display device displays a representation of variants. Finally, a processor is configured to create a data record in the database for each of the multiple short variants and identify for each of the short variant coordinates one of the multiple long variants. The processor then adds to the data record of that short variant a reference to the identified one of the multiple long variants. The processor also generates a user interface with a representation of the multiple short variants that comprise long variant data of the long variant according to the reference from the data record for each of the multiple short variants.

### CROSS-REFERENCE TO RELATED APPLICATIONS

The present application claims priority from Australian Provisional Patent Application No 2016903841 filed on 22 Sep. 2016, the content of which is incorporated herein by reference.

### TECHNICAL FIELD

This disclosure relates to devices, methods and systems for presenting whole genome sequence data.

### BACKGROUND

Genetic testing allows the identification of genetic variants, including mutations, that have an effect on the occurrence of a particular disease or phenotype. In particular, specific loci are known to be associated with particular diseases. For example, the BRCA1 gene is known to be associated with breast cancer and a genetic test is available for this particular locus to assist with predicting a likelihood of developing breast cancer.

Instead of testing at particular loci it is also possible to sequence the entire genome of an individual, which is referred to as Whole Genome Sequencing (WGS). WGS provides more detailed insight into a person's genome than testing at specific loci and allows a more personalised diagnosis or prognosis. However, it is difficult for clinicians, researchers and other users to manually review the large data sets created by WGS. In particular, for professionals who have a practical knowledge of the genome instead of research knowledge it is difficult to use WGS data efficiently in diagnosis or for prognosis.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY

A device for presenting whole genome sequence data of a patient comprises:

a file system to store the whole genome sequence data of the patient, the whole genome sequence data comprising:
a first data file comprising short variant data related to multiple short variants in the patient at respective short variant coordinates;
a second data file comprising long variant data related to multiple long variants in the patient at respective long variant coordinates;

a database to store variant data as data records;

a display device to display a representation of variants; and

a processor configured to
create a data record in the database for each of the multiple short variants,
identify for each of the short variant coordinates one of the multiple long variants where that short variant coordinate lies within the coordinates of the one of the multiple long variants,
add to the data record of that short variant a reference to the identified one of the multiple long variants, and
generate a user interface on the display device, the user interface comprising a representation of the multiple short variants, wherein the representation of the multiple short variants comprises long variant data of the long variant according to the reference from the data record for each of the multiple short variants.

It is an advantage that a clinical practitioner can view the user interface and can see the multiple short variants together with the references to the long variants. This provides a more useful tool to the practitioner as it allows the combination of two separate data sources into a single view. This way, the practitioner can more efficiently peruse the genomic variations and provide a diagnosis more accurately.

The processor may be further configured to execute a short variant calling tool to generate the first data file and a long variant calling tool to generate the second data file.

The long variant calling tool may generate annotation data for each long variant and the reference to the long variant comprises the annotation data.

The processor may be further configured to:

repeat the step of executing a long variant calling tool for multiple different long variant calling tools to generate multiple second data files; and

repeat the steps of identifying one of the multiple long variants and adding to the data record for each of the multiple second data files.

The reference to the long variant may comprise a concatenation of the annotation data from the multiple long variant calling tools.

The database may comprise a long variant table to store long variants from the multiple long variant calling tools as separate rows.

The processor may be further configured to:

identify an inversion in the whole genome sequence data based on the long variant data; and

create two data records in the database to represent the inversion.

The processor may be further configured to:

identify a translocation in the whole genome sequence data based on the long variant data; and

create two data records in the database to represent the translocation.

Creating two data records may comprise creating a link between the two data records.

The database may be a relational database comprising a table to store links between the two data records.

The database may comprise a short variant table to store short variants and a long variant table to store long variants and a sample identifier of the whole genome sequence data serves as a common key between the short variant table and the long variant table.

The database may comprise a gene table to store gene information, wherein the gene information comprises a gene identifier and gene coordinates.

The short variant table may comprise short variant coordinates and the long variant table comprises long variant coordinates and the short variant coordinates, long variant coordinates and gene coordinates serve as a comment key between the short variant table, the long variant table and the gene table.

The processor may be further configured to filter the short variant data based on the long variant data.

The processor may be further configured to filter the short variant data based on an overlap between long variants of different samples and/or long variant calling tools.

The processor may be further configured to filter the short variant data based on Mendelian inheritance associated with the genomic data.

The processor may be further configured to filter the short variant data based on copy number data associated with the long variant data.

A method for presenting whole genome sequence data of an individual comprises:

receiving the whole genome sequence data of the individual, the whole genome sequence data comprising:
short variant data related to multiple short variants of the individual at respective short variant coordinates; and
long variant data related to multiple long variants of the individual at respective long variant coordinates;

identifying for each of the short variant coordinates one of the multiple long variants where that short variant coordinate lies within the coordinates of the one of the multiple long variants;

creating an association between that short variant and the identified one of the multiple long variants; and

generating user interface data, the user interface data comprising a representation of each of the multiple short variants, wherein the representation of each of the multiple short variants comprises long variant data of the identified long variant associated with that short variant.

Software, when installed on a computer, causes the computer to perform the above method.

A computer system for presenting whole genome sequence data of an individual comprises:

a data port to receive the whole genome sequence data of the individual, the whole genome sequence data comprising:
short variant data related to multiple short variants of the individual at respective short variant coordinates; and
long variant data related to multiple long variants of the individual at respective long variant coordinates; and

a processor to:
identify for each of the short variant coordinates one of the multiple long variants where that short variant coordinate lies within the coordinates of the one of the multiple long variants;
create an association between that short variant and the identified one of the multiple long variants; and
generate user interface data, the user interface data comprising a representation of each of the multiple short variants, wherein the representation of each of the multiple short variants comprises long variant data of the identified long variant associated with that short variant.

Optional features described of any aspect of method, computer readable medium or computer system, where appropriate, similarly apply to the other aspects also described here.

### BRIEF DESCRIPTION OF DRAWINGS

An example will be described with reference to

FIG. 1 illustrates a device for presenting whole genome sequence data.

FIG. 2 illustrates a relational database for storing whole genome sequencing data.

FIG. 3 illustrates a method for presenting whole genome sequence data.

FIG. 4 illustrates a resulting short variant table.

FIG. 5 illustrates a user interface presenting whole genome sequence data.

FIG. 6 illustrates a user interface comprising multiple search options.

FIG. 7 illustrates overlapping variants.

### DESCRIPTION OF EMBODIMENTS

Whole genome sequencing (WGS) has become more accessible due to a rapidly falling price tag and a shortened sequencing time facilitated by next generation sequencing (NGS) technologies. The large data sets from sequencers, such as Illumina X10, are analysed by bioinformatics software which align sequence reads to a reference genome, to identify variants, that is, differences between a reference genome and sequences of a sample genome, and which then predict effects of the detected variants on the patient. The outcome may be a prediction of an occurrence or risk of a particular disease or other traits, such as quantitative traits.

Most bioinformatics software tools are designed for specific purposes. Therefore, the output of multiple tools may be combined to arrive at a meaningful result. Some tools generate an output that can be processed by the next tool in the pipeline. In this case, the intermediate result is often of little relevance to the practical application. In other cases, multiple tools are used in parallel to obtain different outputs which are all relevant to the practical application. In particular, when the WGS data is reviewed by a human interpreter, such as a clinical pathologist, the data from multiple tools is reviewed and presented to the interpreter. This presents the difficulty that correlations between the outputs from the different tools are difficult to see. For example, it is difficult to see that a short variant in the output of a short variant caller is within a long variant in the output of a long variant caller. Identifying this relationship would enable the interpreter to draw a conclusion that would be difficult to obtain based on the short variants and long variants in isolation.

While some examples herein relate to medical applications where users of the system include clinical pathologists reviewing patient WGS data, it is to be understood that other applications are equally possible, including lifestyle genomics where personal WGS data is reviewed for specific traits, or veterinary applications including animal breeding and artificial selection where the WGS data relates to individual animals.

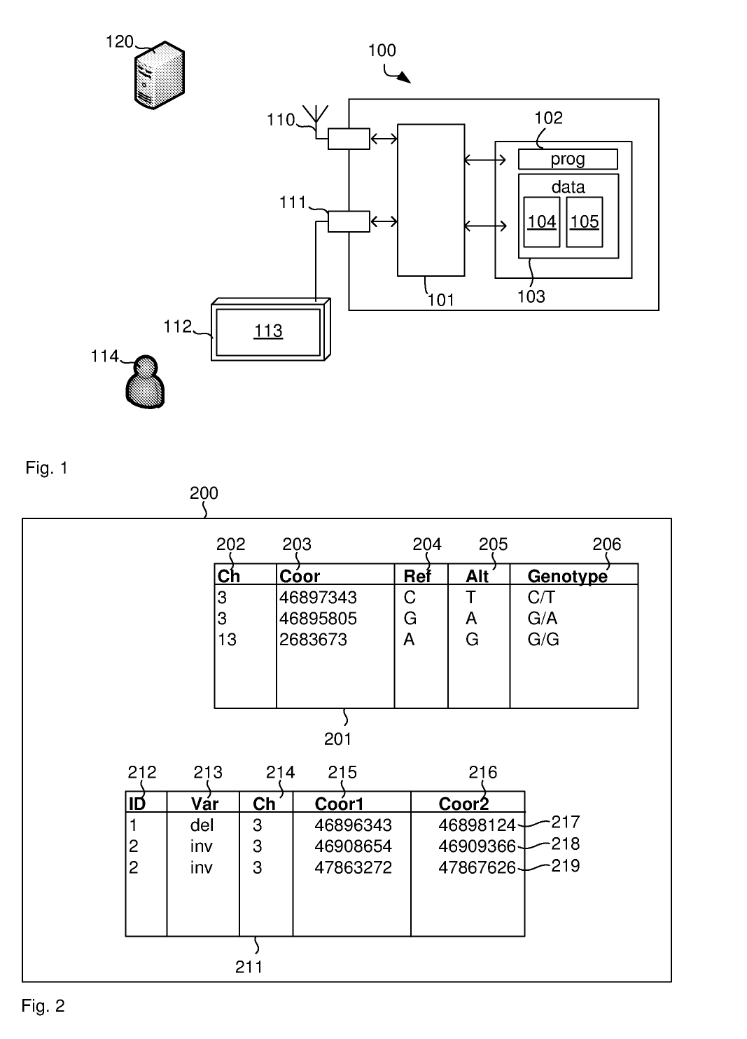

FIG. 1 illustrates a device 100 for presenting whole genome sequence data of a patient such that a relationship between short variants and long variants becomes visible. The computer system 100 comprises a processor 101 connected to a program memory 102, a data memory 103, a communication port 110 and a user port 111. The data memory 103 holds a file system, such as NTFS, FAT32, ext2/ext3/ext4 or others. This file system stores the whole genome sequence data of the patient.

The whole genome sequence data comprises a short variant data file 104 on the file system. The short variant data file 104 comprises short variant data related to multiple short variants of the patient at respective short variant coordinates. For example, the short variant data file 104 may be the output file generated by a short variant calling tool. Tools include, but are not limited to, one or more of GATK HaplotypeCaller, SAMtools mpileup, MuTect and Strelka.

A short variant is a region within a sequenced genome having a sequence that differs from the corresponding region of a reference genome. The reference genome may be a third party reference genome (germline variant) or may be a combination of the latter and a germline genome when sequencing tumour/somatic samples. In the latter case, called “somatic variant”, the short variants are effectively the differences between the germline genome and the tumour/somatic genome. A short variant is typically between 1 and 100 bases in length. A short variant may be a Single Nucleotide Polymorphism (SNP), which is a difference between the sample genome and reference genome at one single locus, or a insertion/deletion (indel) where one or more bases are inserted or deleted from the sample genome relative to the reference genome. Each short variant is located at a short variant coordinate, which is also stored in the short variant data. The coordinate may comprise a chromosome number and the number of bases from the start of the chromosome of the reference genome or the sample genome. For example, the rs6311 variant is a SNP located in chromosome 13 and has the coordinate 13:46897343. The short variant data file may be a text file comprising a string for the SNP type, such as “C/T” for a change from cytosine to thymine and a string “13:46897343” or two numbers “13” and “46897343” for chromosome and base count from start, respectively. The data may be stored in VCF, XML, JSON or other formats including compressed, uncompressed, encrypted and unencrypted formats.

Processor 101 reads the short variant data file and may create a record in a database for each short variant. For example, the database may be a relational database, such as SQL.

FIG. 2 illustrates a relational database 200 for storing whole genome sequencing data hosted on data store 103. Database 200 comprises a short variant table 201 comprising one record for each short variant. In this example, the short variant table 201 has a first data field 202 for chromosome number, a second data field 203 for the coordinate within the chromosome, a third data field 204 for the reference base, a fourth data field 205 for the alternative allele and a fifth data field 206 for the variant genotype. In the example of FIG. 2, there are three short variants, that is, three SNPs in the whole genome sequencing data for this individual.

The whole genome sequence data further comprises a long variant data file 105 on the file system 103. The long variant data file 105 comprises long variant data related to multiple long variants in the individual at respective long variant coordinates. For example, the second data file 105 may be the output file generated by one or more long variant calling tools. Long variant calling tools include, but are not limited to, one or more of CNVnator, PLINK Delly, Sequenza, BreakDancer, Manta and LUMPY.

A long variant is a region of long length within a sample genome that has been affected by a structural and/or copy number genetic variation event, or is otherwise of interest due to being affected by a normal genomic process such as recombination. A long variant ranges in size from 100 bases to hundreds of millions of bases (entire chromosomes). Similar to short variants, long variants may be somatic. That is, long variants may indicate a difference between a tumour/somatic sample and a germline sample.

A long variant may be a structural variant (SV), a copy number variant (CNV) or any region of the genome affected by a genetic process of interest. A long variant (CNV) may be a duplication/deletion. A long variant (CNV) may be an insertion. A long variant (SV) may be an inversion. A long variant (SV) may be a translocation. A region of interest may be a region of homozygosity potentially caused by consanguinity or deletion followed by duplication events in cancer.

Processor 101 reads the long variant data file and may create records in database 200 for the long variants. In one example, processor 101 creates two records for each long variant in a long variant table 211 comprising data fields for block identifier 212, variant type 213, chromosome number 214, a first coordinate 215 and a second coordinate 216.

In the example of FIG. 2, database 200 stores a first record 217 in long variant table 211 which relates to a deletion as indicated by the “del” value in the variant data field 213. This means, the genetic information between the first coordinate 215 and the second coordinate 216 is deleted. For copy number variants and other long variants a single record in long variant table 211 may be sufficient.

Since structural variants may only impact the break points at which they occur, and not the internal sequence, these variants can be represented by two separate records in long variant table 211. For example, database 200 stores a second record 218 and a third record 219 to represent a single structural variation. The first data record 218 represents the imprecise start coordinates of an inversion and the second data record 219 represents the imprecise end coordinates of the inversion. In other words, for this individual, the region between 46908654 and 47867626 on chromosome 3 is inverted. Processor 101 identifies the inversion by reading the output file from the long variant calling tool and creates a link between the two data records 218 and 219 by storing a common identifier ‘2’ in identifier field 212. The link may also be stored in a separate link table having a block identifier field and an event identifier field. The block identifier field is a foreign key to block identifier field 212 of long variant table 211 while the event identifier field is a foreign key to a separate event table. In that case, the link table may have further data fields for long variant data that is associated with each long variant, such that the long variant data is not duplicated in the two entries of the long variant table 211. In particular, the link table may have a data field for variant type instead of variant type data field 213 in long variant table 211. Similarly, processor 101 stores long variant data representing a translocation as two records with a corresponding link.

It is noted that while in the above example the data files 104 and 105 are stored on data store 103 they may equally be stored elsewhere. In particular, data files 104 and 105 may be stored on cloud storage associated with a cloud computing platform that hosts the short variant calling tool(s) and the long variant calling tool(s). For example, DNANexus may be used to execute calling tools on dynamically provisioned virtual machines and to store output files on cloud storage. Processor 101 may then receive the short variant data and long variant data over the Internet or the cloud-internal network. Equally, database 200 may be stored on cloud storage or may be a distributed database. Processor 101 can create, modify and select records in the database remotely by a remote database connection.

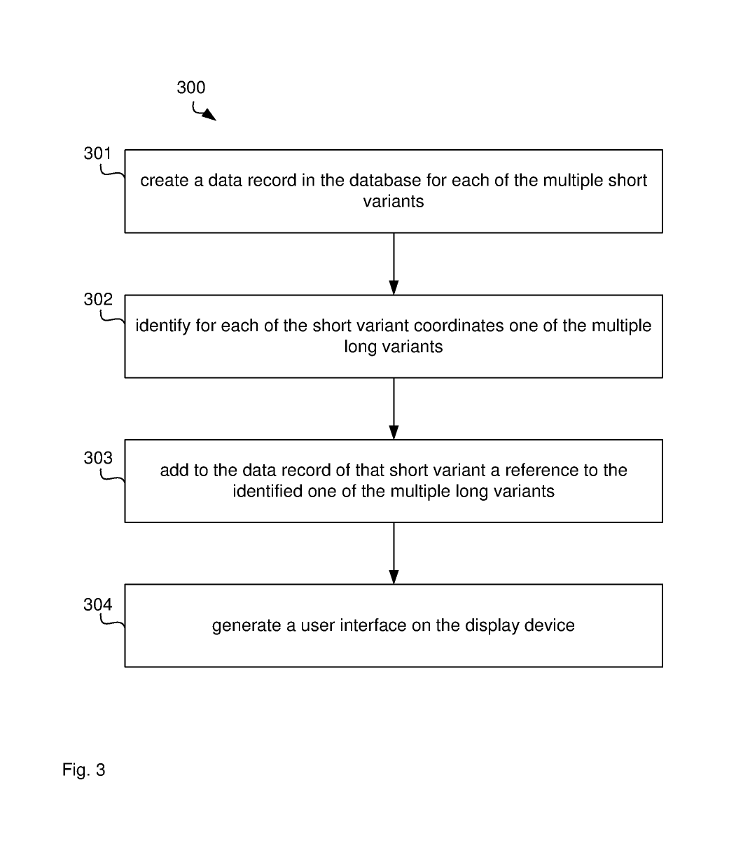

Returning back to FIG. 1, computer system 100 further comprises a display device 112 to display a representation 113 of the variants stored on data store 103 to a user 114. The program memory 102 is a non-transitory computer readable medium, such as a hard drive, a solid state disk or CD-ROM. Software, that is, an executable program stored on program memory 102 causes the processor 101 to perform the method in FIG. 3, that is, processor 101 creates short variant records, identifies one long variant having a short variant within, adds a reference to the long variant and generates a user interface.

The processor 101 may then store the genome data on data store 103, such as on RAM or a processor register. Processor 101 may also send the determined variants via communication port 110 to a server, such as a hospital's patient record server. The processor 101 may receive data, such as WGS data, from data memory 103 as well as from the communications port 110. Processor 101 may receive WGS data from a DNA sequencing machine, such as an Illumina X10. This receiving step may comprise the sequencing machine storing the WGS data on cloud storage and processor 101 retrieving this data from the cloud storage.

Although communications port 110 and user port 111 are shown as distinct entities, it is to be understood that any kind of data port may be used to receive data, such as a network connection, a memory interface, a pin of the chip package of processor 101, or logical ports, such as IP sockets or parameters of functions stored on program memory 102 and executed by processor 101. These parameters may be stored on data memory 103 and may be handled by-value or by-reference, that is, as a pointer, in the source code.

The processor 101 may receive data through all these interfaces, which includes memory access of volatile memory, such as cache or RAM, or non-volatile memory, such as an optical disk drive, hard disk drive, storage server or cloud storage. The computer system 100 may further be implemented within a cloud computing environment, such as a managed group of interconnected servers hosting a dynamic number of virtual machines.

It is to be understood that any receiving step may be preceded by the processor 101 determining or computing the data that is later received. For example, the processor 101 determines WGS data and stores that data in data memory 103, such as RAM or a processor register. The processor 101 then requests the data from the data memory 103, such as by providing a read signal together with a memory address. The data memory 103 provides the data as a voltage signal on a physical bit line and the processor 101 receives the whole genome data via a memory interface.

It is to be understood that throughout this disclosure unless stated otherwise, nodes, edges, graphs, solutions, variables, records, variants, coordinates and the like refer to data structures, which are physically stored on data memory 103 or processed by processor 101. Further, for the sake of brevity when reference is made to particular variable names, such as “coordinate” or “variant” this is to be understood to refer to values of variables stored as physical data in computer system 100.

FIG. 3 illustrates a method 300 as performed by processor 101 for presenting WGS data of a patient. FIG. 3 is to be understood as a blueprint for the software program and may be implemented step-by-step, such that each step in FIG. 3 is represented by a function in a programming language, such as PHP, C++ or Java. The resulting source code may then compiled and stored as computer executable instructions on program memory 102 or in the case of PHP or JavaScript stored directly as computer executable instructions on program memory 102 without compilation.

Processor 101 creates 301 a data record in the database 200 for each of the multiple short variants as described above with reference to FIG. 2. Then, processor 101 identifies 302 for each of the short variant coordinates one of the multiple long variants where that short variant coordinate lies within the coordinates of the one of the multiple long variants. In one example, processor 101 executes two nested loops where the outer loop iterates over all short variants in short variant table 201 and the inner loop iterates over all long variant identifiers in long variant table 211 for the current short variant from the outer loop. Processor 101 checks whether the current short variant coordinate 202 is greater or equal than the start coordinate in first record 215 and less than or equal to the end coordinate in second record 216 of the current long variant. If this comparison is true, processor adds 303 to the data record of that short variant a reference to the identified one of the multiple long variants.

In another example, processor 101 sorts the short variants and the long variants by coordinate. This way, the processor 101 can abort the search earlier and commence the search in the long variant table where it stopped for the previous short variant to accelerate the process.

In yet another example, processor 101 performs a database function, such as a JOIN function based on the coordinates to exploit the optimised database routines. In particular, these coordinates are used as the INNER JOIN condition for searching the blocks. Database 200 stores a genes table with records that link genes to coordinates where each gene->coordinates event has an ID. Processor 101 queries this table for a gene list, which returns all the gene->coordinate IDs. These IDs can then be used to search the block table 211 where the start and end of the block overlaps at all with the coordinates of each of the gene->coordinate IDs returned before. This overlap condition may be included as a WHERE clause into the SELECT statement.

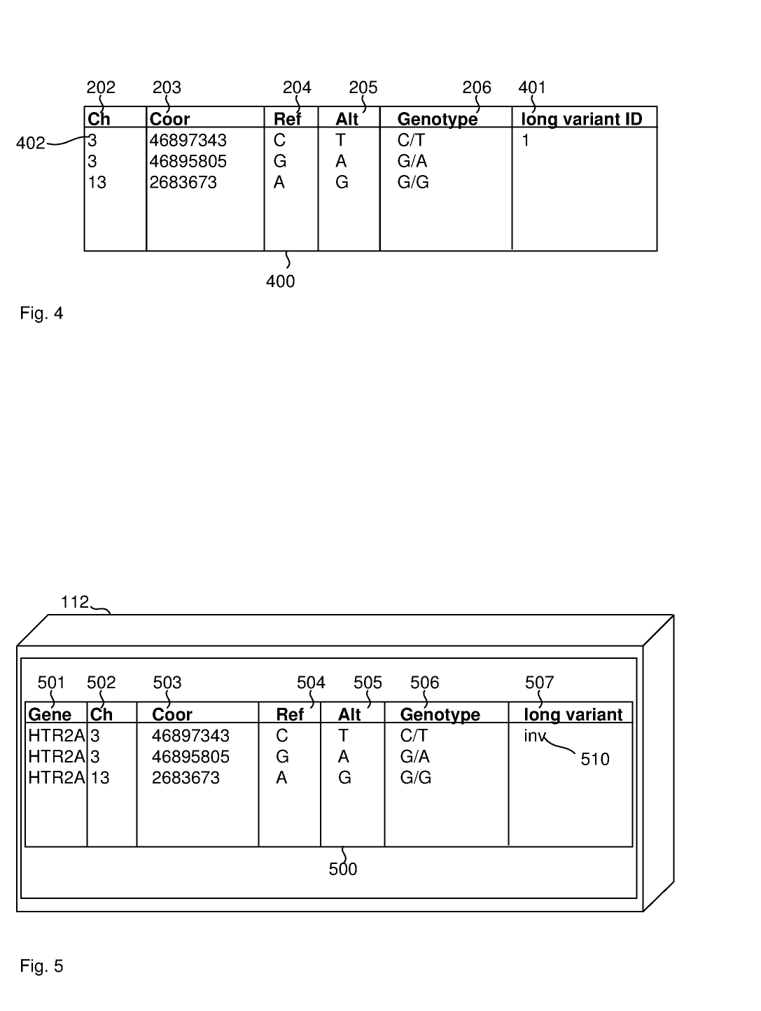

FIG. 4 illustrates a resulting short variant table 400 comprising the data fields from short variant table 201 in FIG. 2 for chromosome number 202, coordinate within the chromosome 203, reference base 204, alternative allele 205 and variant genotype 206. In addition, short variant table 400 now comprises a long variant ID field 401. In this example, processor 101 determines that short variant coordinate 46897343 is greater than long variant start coordinate 46896343 and less than long variant end coordinate 46898124. Therefore, processor 101 adds to data record 402 of this short variant a reference to the identified long variant by including the identifier ‘1’ of the long variant in long variant table 211. This way, processor 101 creates an association between the short variant and the identified long variant. In SQL terms, processor 101 enters a foreign reference into table 400 and the foreign reference relates to a long variant. It is noted that table 400 does not need to be a table in the database but can be a table on a user interface as explained below. In that case, the long variant ID field 401 may contain more information about the long variant than only the reference identifier.

FIG. 5 illustrates a user interface 500 presenting whole genome sequence data which the processor 101 generates 304 on the display device. The user interface 500 comprises a representation of the multiple short variants. For example, the representation may be a list of the multiple short variants. The representation may be a table 500 of the multiple short variants. The representation of the multiple short variants comprises long variant data of the long variant according to the reference from the data record for each of the multiple short variants. In other words, processor 101 retrieves the short variant data from table 400 and for each short variant, processor 101 retrieves the long variant data using the identifier in the long variant ID field 401 as a key. Processor 101 then includes the long variant data into the representation.

Generating the user interface may comprise generating user interface data, such as by writing HTML code to a HTML file that is later rendered remotely by an internet browser. Generating the user interface may also comprise sending user interface data directly to the browser, such as through JavaScript methods. This may include the use of GET and POST methods and XMLHttpRequest data. For example, the JavaScript method may send filter settings and request a list of short variants to a Software as a Service (SaaS) platform. The SaaS platform responds by sending the list of short variants where each item in the list is a representation of a short variant and may include the long variant data. The JavaScript method can then iterate over the received list object and create a table row for each item in the list object. This may be performed within an AJAX framework or an Angular frontend connected to a Flask backend.

In the example of FIG. 5 table 500 of the short variants comprises a gene name column 501, a chromosome column 502, a coordinate column 503, a reference base column 504, an alternative allele column 505, a genotype column and a long variant data column 507. Table 500 may comprise a locus name column in addition to or instead of the gene name column 501 for situations where a region in the genome is defined and labelled by a name but a gene is not known or not directly associated with that region. In the example of FIG. 5, only the first variant 510 was found in a long variant coordinate range. As a result, processor 101 adds long variant data into column 507. In this example, processor 101 adds the string ‘inv’ from table 211 in FIG. 2 to indicate to the user that variant 510 is located within a region that is also the subject of an inversion event.

Database 200 may comprise a separate gene table. This gene table comprises data fields for a gene identifier, such as “BRCA1” and the corresponding gene coordinates including a start and an end coordinate. The gene table may comprise a data field for a gene description, associated diseases and other information. Processor 101 may query the gene table when generating the user interface table 500 and include the gene information into the table in the gene column 501. In order to optimise performance, processor 101 may perform an SQL JOIN statement between the gene table, the long variant table and the short variant table with the coordinates as the common key.

It is noted that table 500 may contain more or less columns than shown in FIG. 5. For example, table 500 may not have the coordinate column 503 in applications where users are unlikely to be able to interpret the large numbers typically associated with coordinates. On the other hand, table 500 may comprise further columns indicative of associations between a short variant and a disease or other traits or phenotypes.

In one example, long variant data column 507 shows the entire output generated by the long variant calling tool for the identified long variant, such as the coordinate range.

A user, such as a clinical pathologist, can then review the list of short variants and can conveniently see for each short variant whether that short variant is also nested within a long variant, such as a structural variant. This allows the user to draw more accurate conclusions from the WGS data, such as a more accurate diagnosis. In cases where only a small number of qualified users are available for a large number of patients, the proposed system allows the user to perform their duties more efficiently and help more patients than otherwise possible.

Processor 101 may execute multiple different long variant calling tools to generate multiple long variant data files. This may be useful when there are multiple long variant calling tools available and each tool has particular advantages or can call different types of long variants. In this case, processor 101 repeats the steps of identifying 302 for each one of the multiple long variants and adding 303 to the data record for each of the multiple second data files. Long variant data column 507 in FIG. 5 may then comprise a concatenation of the output data from the different long variant calling tools.

Processor 101 may also generate a filter interface on display device 112 to allow the user to reduce the number of short variants that are displayed in representation 500. The filter interface may comprise multiple different filters. The filters may comprise a gene name filter where a user can enter or select the name of one or more genes and processor 101 includes only variants within the entered or selected one or more genes. More particularly, processor 101 may query the gene table to retrieve all sets of chromosome, start and end coordinates of a selected gene and then determine which variants are within these coordinates. The user may be aware of an association between certain genes and observed traits and therefore, it is useful for the user to limit the output to those genes.

Similarly, the filters may also include a gene coordinate filter such that processor 101 only includes variants that lie within a provided coordinate range.

The filters may also include an overlap filter. In this case, processor 101 determines whether the coordinate range of a long variant overlaps with the coordinate range of any other long variant and only includes those long variants if they overlap. Overlaps may be pairwise, between samples or between long variant types/methods within a given set of samples and variant types/methods.

In one example, the short variant data and the long variant data relate to multiple samples, that is, multiple patients or subjects. In this case, the data tables 201 and 211 may comprise an additional data field for a sample identifier. The sample identifier of the WGS data may then serve as a common key between the short variant table and the long variant table. In other words, processor 101 can group the variants by the sample identifier or only retrieve variants that relate to a particular sample. Further, processor 101 can determine which long variants overlap between samples. This may apply to the use case of a single long variant calling tool and the overlap filter is configured by the user to only show long variants that overlap, which means individuals have long variants at similar positions. This may be useful when investigating inherited traits where the ancestors and the offspring share the same long variant that may be responsible for that trait, such as in the case of a heritable disease.

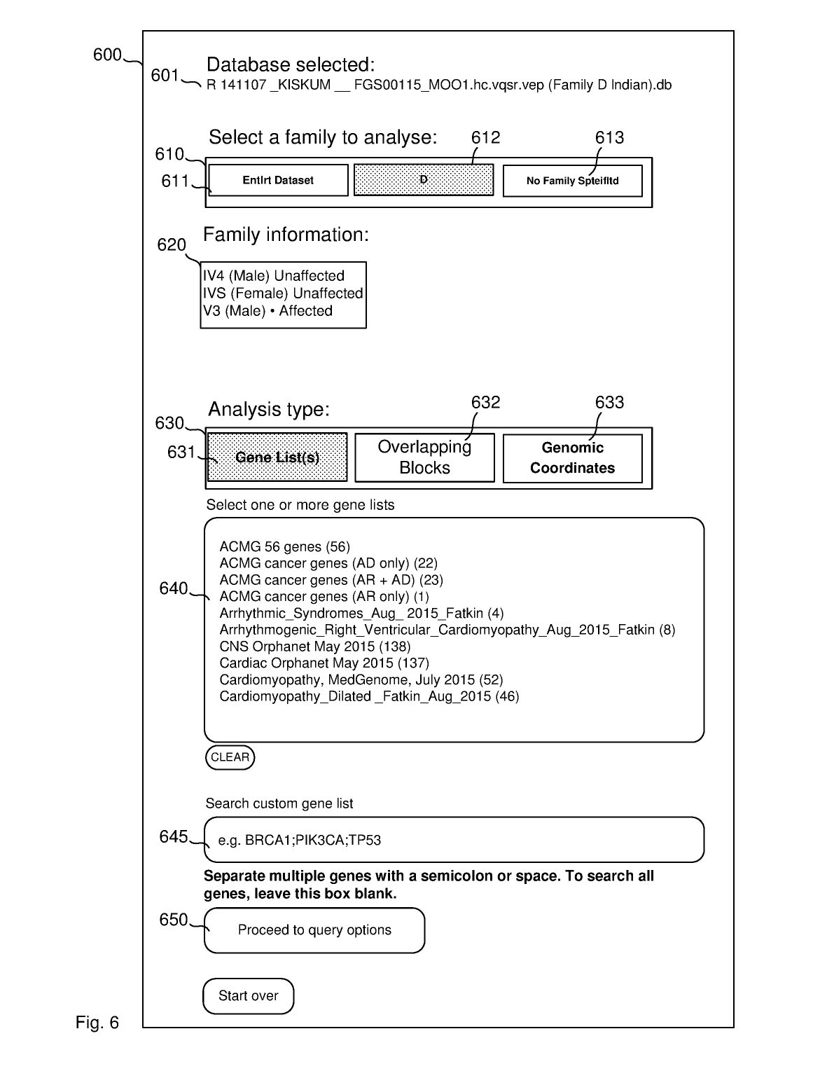

FIG. 6 illustrates a user interface 600 comprising multiple search options. User interface 600 comprises a database identifier 601 to indicate to the user which database is currently selected. It is noted that the database may hold variant data related to multiple individuals, such as multiple family members. User interface 600 further comprises a family selector 610 including options for the entire dataset 611, a particular family 612 labelled ‘D’ or proceed without specifying a family 613. It is noted that in cases where the selected database comprises variant data of multiple families, the selector button 612 would be replicated for each family with a respective label replacing ‘D’ in FIG. 6. Processor 101 receives the selection of the family through selector 610, retrieves family information from the database and displays that information in a family information text field 620, such as for each individual family member whether that individual is affected.

User interface 600 further comprises an analysis type selector 630 where the user can choose between gene lists 631, overlapping blocks 632 and genomic coordinates 633. Ultimately, the goal of these queries is to obtain a list of genomic blocks that match specific criteria for a set of samples. Upon receiving the selection of querying gene lists 631, processor 101 displays all blocks for all selected samples that overlap with any of the genes in one or more gene lists specified. Upon receiving the selection of overlapping blocks 632 processor 101 displays blocks for all selected samples that overlap by one or more bases. Upon receiving the selection of genomic coordinates 633, processor 101 displays blocks for all selected samples where a block overlaps with one or more samples at one or more bases.

User interface 600 further comprises a selectable gene list 640 where a user can select one or more genes from that list. Processor 101 receives the selection from user interface 600 and limits the listed variants to those that fall within the selected genes. User interface 600 also comprises a custom gene list 645 where a user can type or paste gene names directly with the same effect as selecting the genes manually in selectable gene lest 640. A submit button 650 causes the processor 101 to retrieve the entered data from user interface 600, perform the corresponding query and list the resulting variants as described herein.

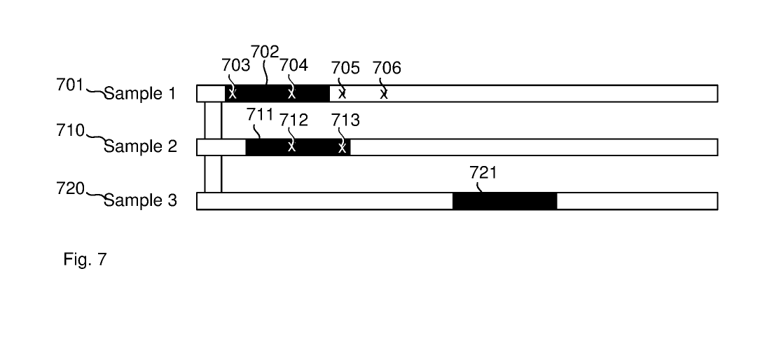

FIG. 7 illustrates overlapping variants in more detail where the horizontal direction represents the gene coordinate. In this example, database 200 stores long variant data and short variant data of three samples. A first sample 701 has a long variant 702 and four short variants 703, 704, 705 and 706, respectively. A second sample 710 has second long variant 711 and two short variants 712 and 713 corresponding to short variants 704 and 705, respectively. In other words, individuals corresponding to samples 701 and 710 share the same short variants 704/712 and 705/713. A third sample 720 has third long variant 721. As can be seen in FIG. 7, first long variant 702 overlaps with second long variant 711. Short variants 703 and 704 are within first long variant 702 but only short variant 704 (as short variant 712) is also within overlapping long variant 711. As a result, activating the overlap filter will cause processor 101 to show only the short variant 704/712 as this short variant is within the region of the long variant 702 that overlaps with another long variant 711 from a different sample. In other words, when restricting variants based on overlaps of blocks, processor 101 returns short variants that are present in both individuals and also in overlapping blocks, i.e. the block was inherited with the short causative variant within it.

Short variants 703, 705 and 706 are not within the region of overlap between long variants 702 and 711 and are therefore excluded from the results. The third long variant 721 does not overlap with any of the other long variants and any short variants (not shown) within third long variant 721 are also excluded. The overlap filter allows the user to view only long variants that are common between different samples, which can reduce the number of variants significantly.

Processor 101 may apply the overlap filter as described above for different long variant calling tools such that the three samples 701, 710 and 720 are replaced by the output of three long variant calling tools.

The long variant data may comprise inheritance data. For example, the long variant table 211 may comprise a data field for inheritance. Inheritance information may be stored with the short variants or stored in a central table separate to both short and long variants. In one example, stored information comprises affected/unaffected status and male/female/unknown gender. Dominant/recessive/compound inheritance predictions may be stored as part of the phenotype data for the patient/family and may be stored in an external database. Data values may include autosomal dominant, autosomal recessive, compound heterozygous and de novo dominant. Processor 101 can then perform an inheritance filter such that only those short variants are shown where the corresponding long variant has a user-specified inheritance value. The inheritance value may be generated by an inheritance analyser, such as GEMINI.

The long variant data may comprise copy number data. For example, the long variant table 211 may comprise a data field for copy number. Data values may be numeric or NULL where no copy number estimate was made. Processor 101 can then perform a copy number filter such that only those short variants are shown where the corresponding long variant has a user-specified copy number. The copy number value may be generated by a long variant detection tool.

By applying these filters in different combinations a user can interactively reduce the number of variants for the particular individual. This allows the user to make full use of the available WGS data and derive conclusions or diagnoses that would otherwise have been difficult if not impossible to derive.

It is noted that processor 101 may also operate on the long variants only without reference to the short variants. In this case, processor 101 may filter the long variants by overlapping long variants from different samples and/or different individuals. For example, a user could ask what are the genes within overlapping blocks of regions of homozygosity in the affected samples in a given family and the output would be long variants and the genes within them only.

It will be appreciated by persons skilled in the art that numerous variations and/or modifications may be made to the specific embodiments without departing from the scope as defined in the claims.

It should be understood that the techniques of the present disclosure might be implemented using a variety of technologies. For example, the methods described herein may be implemented by a series of computer executable instructions residing on a suitable computer readable medium. Suitable computer readable media may include volatile (e.g. RAM) and/or non-volatile (e.g. ROM, disk) memory, carrier waves and transmission media. Exemplary carrier waves may take the form of electrical, electromagnetic or optical signals conveying digital data steams along a local network or a publically accessible network such as the internet.

It should also be understood that, unless specifically stated otherwise as apparent from the following discussion, it is appreciated that throughout the description, discussions utilizing terms such as “estimating” or “processing” or “computing” or “calculating”, “optimizing” or “determining” or “displaying” or “maximising” or the like, refer to the action and processes of a computer system, or similar electronic computing device, that processes and transforms data represented as physical (electronic) quantities within the computer system's registers and memories into other data similarly represented as physical quantities within the computer system memories or registers or other such information storage, transmission or display devices.

The present embodiments are, therefore, to be considered in all respects as illustrative and not restrictive.

1. A device for presenting whole genome sequence data of a patient, the device comprising:
a file system to store the whole genome sequence data of the patient, the whole genome sequence data comprising:
a first data file comprising short variant data related to multiple short variants in the patient at respective short variant coordinates;
a second data file comprising long variant data related to multiple long variants in the patient at respective long variant coordinates;
a database to store variant data as data records;
a display device to display a representation of variants; and
a processor configured to
create a data record in the database for each of the multiple short variants,
identify for each of the short variant coordinates one of the multiple long variants where that short variant coordinate lies within the coordinates of the one of the multiple long variants,
add to the data record of that short variant a reference to the identified one of the multiple long variants, and
generate a user interface on the display device, the user interface comprising a representation of the multiple short variants, wherein the representation of the multiple short variants comprises long variant data of the long variant according to the reference from the data record for each of the multiple short variants.

2. The device of claim 1, wherein the processor is further configured to execute a short variant calling tool to generate the first data file and a long variant calling tool to generate the second data file.

3. The device of claim 2, wherein the long variant calling tool generates annotation data for each long variant and the reference to the long variant comprises the annotation data.

4. The device of claim 3, wherein the processor is further configured to:
repeat the step of executing a long variant calling tool for multiple different long variant calling tools to generate multiple second data files; and
repeat the steps of identifying one of the multiple long variants and adding to the data record for each of the multiple second data files.

5. The device of claim 4, wherein the reference to the long variant comprises a concatenation of the annotation data from the multiple long variant calling tools.

6. The device of claim 4, wherein the database comprises a long variant table to store long variants from the multiple long variant calling tools as separate rows.

7. The device of claim 1, wherein the processor is further configured to:
identify an inversion in the whole genome sequence data based on the long variant data; and
create two data records in the database to represent the inversion.

8. The device of claim 1, wherein the processor is further configured to:
identify a translocation in the whole genome sequence data based on the long variant data; and
create two data records in the database to represent the translocation.

9. The device of claim 7, wherein creating two data records comprises creating a link between the two data records.

10. The device of claim 7, wherein the database is a relational database comprising a table to store links between the two data records.

11. The device of claim 1, wherein the database comprises a short variant table to store short variants and a long variant table to store long variants and a sample identifier of the whole genome sequence data serves as a common key between the short variant table and the long variant table.

12. The device of claim 11, wherein the database comprises a gene table to store gene information, wherein the gene information comprises a gene identifier and gene coordinates.

13. The device of claim 12, wherein the short variant table comprises short variant coordinates and the long variant table comprises long variant coordinates and the short variant coordinates, long variant coordinates and gene coordinates serve as a common key between the short variant table, the long variant table and the gene table.

14. The device of claim 1, wherein the processor is further configured to filter the short variant data based on the long variant data.

15. The device of claim 14, wherein the processor is further configured to filter the short variant data based on an overlap between long variants of different samples and/or long variant calling tools.

16. The device of claim 14, wherein the processor is further configured to filter the short variant data based on Mendelian inheritance associated with the genomic data.

17. The device of claim 14, wherein the processor is further configured to filter the short variant data based on copy number data associated with the long variant data.

18. A method for presenting whole genome sequence data of an individual, the method comprising:
receiving the whole genome sequence data of the individual, the whole genome sequence data comprising:
short variant data related to multiple short variants of the individual at respective short variant coordinates; and
long variant data related to multiple long variants of the individual at respective long variant coordinates;
identifying for each of the short variant coordinates one of the multiple long variants where that short variant coordinate lies within the coordinates of the one of the multiple long variants;
creating an association between that short variant and the identified one of the multiple long variants; and
generating user interface data, the user interface data comprising a representation of each of the multiple short variants, wherein the representation of each of the multiple short variants comprises long variant data of the identified long variant associated with that short variant.

19. Software that, when installed on a computer, causes the computer to perform the steps of:
receiving the whole genome sequence data of the individual, the whole genome sequence data comprising:
short variant data related to multiple short variants of the individual at respective short variant coordinates; and
long variant data related to multiple long variants of the individual at respective long variant coordinates;
identifying for each of the short variant coordinates one of the multiple long variants where that short variant coordinate lies within the coordinates of the one of the multiple long variants;
creating an association between that short variant and the identified one of the multiple long variants; and
generating user interface data, the user interface data comprising a representation of each of the multiple short variants, wherein the representation of each of the multiple short variants comprises long variant data of the identified long variant associated with that short variant.

20. (canceled)
